(19) United States
(12) Patent Application Publication
(10) Pub. No.: US 2012/0215136 A1
(43) Pub. Date: Aug. 23, 2012

(54) MEDICAL DEVICE

(75) Inventors: John B. Schneider, Memphis, TN (US); Richard H. Pearce, Cary, NC (US); Jin Zhang, Memphis, TN (US)

(73) Assignee: SMITH & NEPHEW, INC., Memphis, TN (US)

(21) Appl. No.: 13/278,560

(22) Filed: Oct. 21, 2011

Related U.S. Application Data
(60) Provisional application No. 61/405,757, filed on Oct. 22, 2010.
(60) Provisional application No. 61/483,445, filed on May 6, 2011.

(51) Int. Cl. A61N 7/00

(52) U.S. Cl. 601/2

(57) ABSTRACT

A modular medical device includes a core module housing. The core module housing includes a treatment module interface configured to couple to any one of a plurality of treatment modules, and each of the plurality of treatment modules includes a treatment assembly configured to apply a medical treatment. The core housing module contains one or more processing devices coupled to the treatment module interface and configured to determine which particular treatment module, from the plurality of treatment modules, is coupled to the treatment module interface. The one or more processing devices are configured to select, based on the determination, a set of control instructions for the particular treatment module from a plurality of control instructions, and control the particular treatment module using the selected control instructions such that the treatment assembly of the particular treatment module applies the medical treatment for which the treatment assembly is configured.

### CROSS-REFERENCE TO RELATED APPLICATIONS

This application claims priority to and the full benefit of U.S. Provisional Application Ser. No. 61/405,757, filed Oct. 22, 2010, and titled “Medical Device,” and U.S. Provisional Application Ser. No. 61/483,445, filed May 6, 2011, and titled “Medical Device,” the entire contents of which are incorporated herein by reference.

### TECHNICAL FIELD

This description relates to a medical device.

### BACKGROUND

Medical devices can provide treatment for a variety of health conditions. In some instances, a patient has a degree of control over treatment with a medical device. For example, a patient may be able to initiate treatment with a medical device. The capabilities of a medical device determine to a large degree the way that the patient and others interact with the medical device. In particular, it is important that a medical device be capable of providing effective treatment and a positive patient experience.

### SUMMARY

In one general aspect, a modular medical device includes a core module housing. The core module housing includes a treatment module interface configured to couple to any one of a plurality of treatment modules, and each of the plurality of treatment modules includes a treatment assembly configured to apply a medical treatment. The core housing module contains one or more processing devices coupled to the treatment module interface and configured to determine which particular treatment module, from the plurality of treatment modules, is coupled to the treatment module interface when one of the treatment modules is coupled to the treatment module interface. The one or more processing devices are configured to select, based on the determination, a set of control instructions for the particular treatment module from a plurality of control instructions, and control the particular treatment module using the selected control instructions such that the treatment assembly of the particular treatment module applies the medical treatment for which the treatment assembly is configured.

Implementations may include one or more of the following features. For example, the treatment assemblies of the plurality of treatment modules are configured to treat different health conditions. The treatment assembly of each of the plurality of treatment modules includes an ultrasound assembly configured to apply an ultrasound treatment. To control the particular treatment module, the one or more processing devices are configured to control the particular treatment module using the selected control instructions such that the ultrasound assembly of the particular treatment module applies the ultrasound treatment for which the ultrasound assembly is configured. Each of the ultrasound assemblies includes one or more ultrasound transducers and one or more ultrasound transducer driver circuits. The one or more ultrasound transducers of each of the plurality of treatment modules vary in number, size, angle, or application method to treat a health condition. The core module contains one or more data storage devices in communication with the one or more processing devices. To select the set of control instructions for the particular treatment module from the plurality of control instructions, the one or more processing devices are configured to access information stored on the data storage device that indicates a frequency, an intensity, or a duration of ultrasound to be produced by the particular treatment module. The core module contains one or more data storage devices in communication with the one or more processing devices, the data storage device storing module profiles for each of the plurality of treatment modules. The core module housing further contains a power supply module configured to supply power over the treatment module interface. Each of the plurality of treatment modules includes a treatment module identifier, and the one or more processing devices are further configured to receive the treatment module identifier from the particular treatment module. To determine which treatment module is coupled to the treatment module interface, the one or more processing devices are configured to match the received treatment module identifier with a stored treatment module identifier. Each of the plurality of treatment modules includes an applicator configured for treatment of a different health condition. The core module housing further contains a LCD screen and a wireless transceiver.

In another general aspect, a method performed by one or more processing devices includes determining, by a processing device in a core module housing, which treatment module of a plurality of treatment modules is coupled to a treatment module interface of the core module housing, each of the plurality of treatment modules including a treatment assembly configured to apply a medical treatment. The computer-implemented method includes selecting, based on the determination, a set of control instructions for the particular treatment module from a plurality of control instructions, and controlling the particular treatment module using the selected control instructions such that the treatment assembly of the particular treatment module applies the medical treatment for which the ultrasound assembly is configured.

Implementations may include one or more of the following features. For example, the treatment assemblies of the plurality of treatment modules are configured to treat different health conditions. Each of the plurality of treatment modules includes an ultrasound assembly configured to apply an ultrasound treatment for a particular health condition. Controlling the particular treatment module using the selected control instructions includes controlling the particular treatment module using the selected control instructions such that the ultrasound assembly of the particular treatment module applies the ultrasound treatment for which the ultrasound assembly is configured. Each of the ultrasound assemblies includes one or more ultrasound transducers and one or more ultrasound transducer driver circuits. The one or more ultrasound transducers of each of the plurality of treatment modules vary in number, size, angle, or application method to treat a particular health condition. Selecting a set of control instructions for the particular treatment module from the plurality of control instructions includes accessing information stored on a data storage device of the core module housing that indicates a frequency, an intensity, or a duration of ultrasound to be produced by the particular treatment module. The computer-implemented method includes supplying power to the particular treatment module over the treatment module interface. Determining which treatment module of the plurality of treatment modules is coupled to the treatment module interface includes receiving a treatment module identifier from the particular treatment module. Determining which treatment module of the plurality of treatment modules is coupled to the treatment module interface includes matching the received treatment module identifier with a stored treatment module identifier. Each of the plurality of treatment modules includes an applicator configured for treatment of a different health condition.

In another general aspect, a modular medical device for ultrasound therapy includes a core module housing. The core module housing contains a treatment module interface configured to couple to any one of a plurality of treatment modules. Each of the plurality of treatment modules includes an ultrasound assembly configured to apply an ultrasound treatment for a particular health condition. The core module housing contains one or more processing devices coupled to the treatment module interface and configured to, when one of the treatment modules is coupled to the treatment module interface, determine which particular treatment module, from the plurality of treatment modules, is coupled to the treatment module interface. The one or more processing devices are configured to select, based on the determination, a set of control instructions for the particular treatment module from the plurality of control instructions, and control the particular treatment module using the selected control instructions such that the ultrasound assembly of the particular treatment module applies the ultrasound treatment for which the ultrasound assembly is configured.

In another general aspect, a method performed by one or more processing devices includes determining, by a processing device in a core module housing, which treatment module of a plurality of treatment modules is coupled to a treatment module interface of the core module housing, each of the plurality of treatment modules including an ultrasound assembly configured to apply an ultrasound treatment for a particular health condition. The computer-implemented method includes selecting, based on the determination, a set of control instructions for the particular treatment module from a plurality of control instructions, and controlling the particular treatment module using the selected control instructions such that an ultrasound assembly of the particular treatment module applies the ultrasound treatment for which the ultrasound assembly is configured.

The details of one or more implementations are set forth in the accompanying drawings and the description below. Other features and advantages will become apparent from the description, the drawings, and the claims.

### DESCRIPTION OF DRAWINGS

FIG. 1 is a perspective view of a medical device.

FIG. 2 is a block diagram of the medical device.

FIGS. 3A and 3B are perspective views of modular medical devices.

FIG. 4 is a diagram of a modular medical device.

FIGS. 5A to 5D are perspective diagrams of applicators for a modular medical device.

### DETAILED DESCRIPTION

In some implementations, a medical device can be coupled to one or more removable modules. For example, the medical device can include a control unit that can control multiple treatment modules, and various treatment modules may provide treatment for different health conditions. A first treatment module may be used to treat a first health condition, and the first treatment module may be replaced with a second treatment module to treat a second health condition. As a result, a single control unit may be used to provide different types of treatment, even when different treatments require physically different treatment components. Other modules can supplement the display or communication capabilities of a medical device.

Some implementations of the medical device may provide the following advantages. For example, because the medical device can operate with various treatment modules, a patient can use the medical device to treat a wide variety of health conditions. A patient can expand the capabilities of the medical device without replacing the entire medical device. Modules of the medical device can be replaced in the event of wear or damage without requiring replacement of the control unit or functioning modules. Modular design also enables the manufacturer of the medical device to provide medical devices with many different combinations of features. Low-cost medical devices may omit optional modules to reduce cost, while high-end medical devices can include optional modules to enhance the patient's experience.

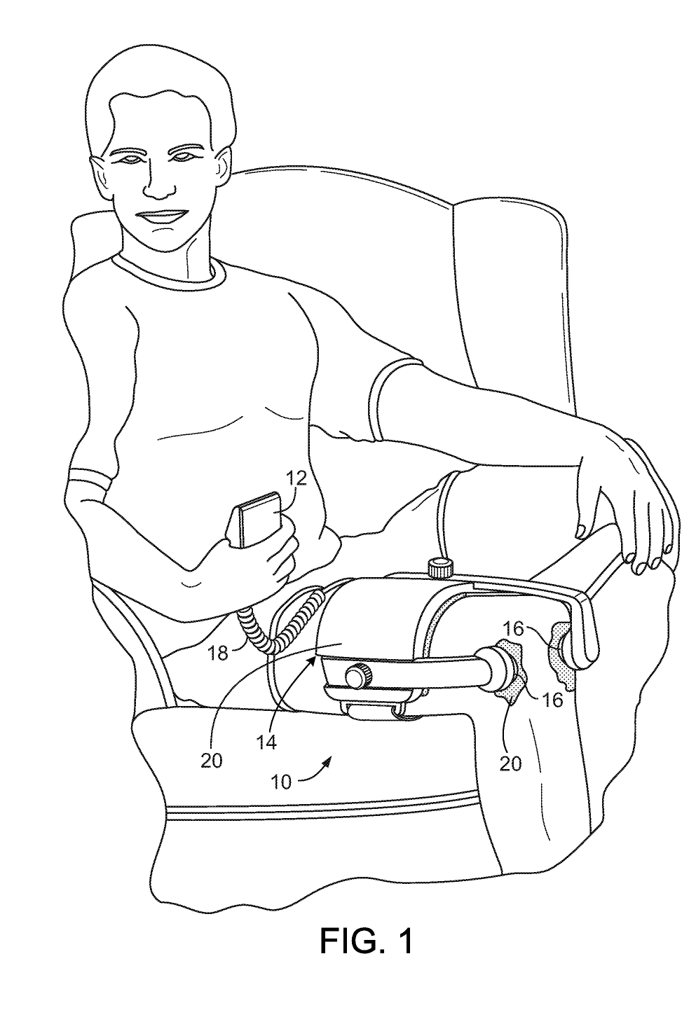

Referring to FIG. 1, a patient is shown using a medical device 10 that includes a treatment module for applying a treatment to the patient. The medical device 10 is a portable ultrasonic treatment device that is equipped to provide, for example, purchasable treatments.

The treatment module may include, for example, one or more ultrasound transducers 16 and at least one driver circuit coupled to the ultrasound transducers 16.

The medical device 10 can include a control unit 12 that controls the operation of the transducers 16. The control unit 12 can include the transducer driver circuit. As described further below, a transducer driver circuit can alternatively be located in a removable treatment module. The medical device 10 can also include cables 18 that can carry power, data, and control signals between the control unit 12 and the transducers 16.

The medical device 10 can include a placement module 14 that couples the transducers at a location of the patient's body where treatment is needed, for example, over a fractured bone or next to damaged connective tissue. The placement module 14 can include a band, sleeve, or other connector to fasten the one or more transducers to a treatment site. An ultrasound conducting gel 20 can be applied to the skin of the patient to enable the ultrasound to propagate effectively to the patient's tissue.

The medical device 10 can use low intensity, ultra high-frequency acoustic energy (ultrasound) to treat injuries, defects, or pathologies. For instance, the ultrasonic treatment device can be designed to treat injuries, defects, or pathologies of bones or connective tissue, and, in some instances, can increase vascularization of ischaemic or grafted tissue. The medical device 10 may be used as an adjunct to surgical repair, in order to speed healing, or in some cases can be used alone to heal tissue injuries without surgery (e.g., for degenerative diseases such as osteoarthritis, tendonosis, and tendonitis). The medical device 10 can be suitable for use in treatment of bone fractures and/or connective tissues associated with joints, such as those in the hand, foot, wrist, ankle, knee, elbow, hip, shoulder, back, and neck.

For example, following surgery, the medical device 10 can be applied non-invasively to the outside of the body (e.g., coupled to the skin with coupling media, such as a gel) in the region of the repaired tissue. The medical device 10 can be operated to transmit ultrasound (for example, in the form of pulses) into the tissue in need of treatment, or at the interface with the uninjured tissues. Exposure to the ultrasound can stimulate a faster, better quality repair of the tissue. At a bone interface, the ultrasound can also stimulate bone repair and bone ingrowth into repair or graft tissue. This can give rise to a faster, stronger repair and improved integration of the interface between, for example, tendon, ligament, and bone. The ultrasonic treatment device may also be used to non-invasively treat pathologies of connective tissues, such as osteoarthritis, ligament and tendon conditions, without the need for a surgical procedure.

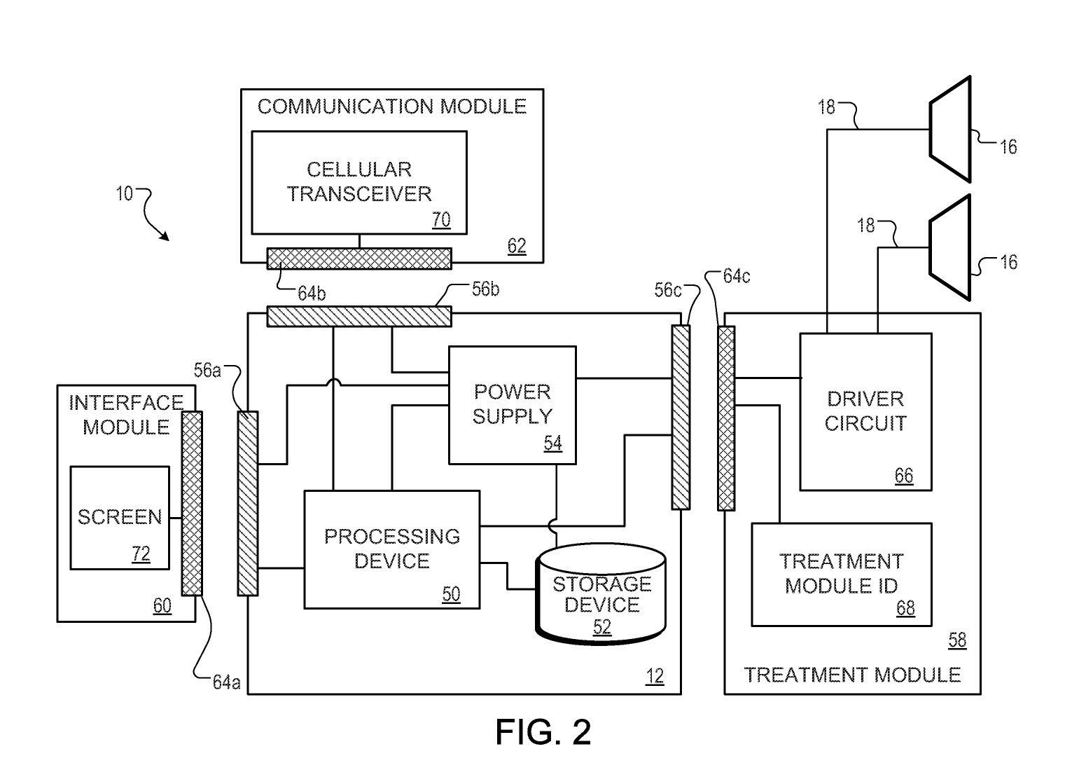

Referring to FIG. 2, the control unit 12 of the medical device 10 can include a processing device 50 that executes instructions stored on a storage device 52. The control unit 12 can also include a power supply 54 and one or more module interfaces 56a-56c. The power supply 54 can be configured to supply power over the interfaces 56a-56c and also to supply power to components of the control unit 12. The control unit 12 can be a core module housing that can receive one or more modules at the module interfaces 56a-56c. One or more modules can be coupled to the interfaces 56a-56c, including, for example, a treatment module 58, interface module 60, and a communication module 62.

The module interfaces 56a-56c can each receive one or more modules. The interfaces 56a-56c can be configured to removably couple one or more modules to the control unit 12. When a module is connected one of the interfaces 56a-56c, an operative connection may be established between the module and the processing device 50, other components of the control unit 12, and/or one or more other modules.

The operative connection between the control unit 12 and a module can enable power, data, and control signals to be exchanged over the interfaces 56a-56c. For example, when a module is connected at an interface 56a-56c, power from the control unit 12 may be supplied to the module over the interface 56a-56c, and communication may be established between the processing device 50 and the module. The module may transmit an identifier to the processing device 50 to identify the type and capabilities of the module and/or other information about the module. The processing device 50 may send control signals to the module to control the operation of the module. The processing device 50 and the module may also exchange other information to enable the full functionality of the medical device 10.

The interfaces 56a-56c may be proprietary or standard. Examples of standard interfaces and protocols include, for example, Universal Serial Bus (USB), Firewire, Subscriber Information Module (SIM), Serial Advanced Technology Attachment (SATA), Secure Digital (SD), Compact Flash, and Ethernet. The operative connections of the interfaces 56 may include one or more combinations of mechanical, magnetic, electrical, or optical connections. In one implementation, a module can be physically coupled to the control unit 12 at the interface 56a-56c.

In one implementation, multiple interfaces 56a-56c may include a common connector, allowing different types of modules to be interchangeably connected at one of several interfaces 56a-56c. In another implementation, an interface 56a-56c may be configured to receive only a particular type of module, and to not connect with other types of modules. For example, a first interface 56a may connect only to one or more communication modules 62, a second interface 56b may connect only to one or more interface modules 60, and a third interface 56c may connect only to one or more treatment modules 58. In this manner, the interfaces 56a-56c can prevent a module from being installed at an incorrect interface 56a-56c.

The interfaces 56a-56c and modules may be configured so that a module is completely or partially housed in the control unit 12 when the module is coupled at an interface 56a-56c. The interfaces 56a-56c may also be configured so that some or all of a module is visible, or extends from the control unit 12, when coupled at an interface 56a-56c. An interface 56a-56c may include one or more sockets, wires, or other connectors that extend from the control unit 12 or are disposed in a housing of the control unit 12.

Examples of modules include the treatment module 58, the communication module 62, and the interface module 60. The power supply 54 may also be implemented as a module. Each module can include an interface 64a-64c that is configured to (i) engage at least one of the interfaces 56a-56c of the control unit 12 and (ii) establish an operative connection between the module and the control unit 12. Each module may be removable and/or replaceable. The medical device 10 may be able to apply treatment with a treatment module 58 even when one or more other modules (for example, the interface module 60 or the communication module 62) are not present.

The treatment module 58 can be configured to apply a treatment to a patient by, for example, producing ultrasound with therapeutic properties. The treatment module 58 can include, for example, one or more ultrasound transducers 16, an ultrasound transducer driver circuit 66, and an embedded module identifier 68, which can be stored on a storage device (not shown). Various treatment modules 58 may include different numbers, shapes, and configurations of transducers 16 in order to treat a variety of health conditions.

In one implementation, a treatment module 58 may be configured to apply treatment for a particular health condition or a particular set of health conditions. For example, one treatment module may include a single transducer 16 to treat a fracture of a small bone. Another treatment module 58 may include two transducers 16 to treat a fracture of a large bone. The treatment module 58 can include an applicator configured for treatment of a particular health condition.

The driver circuit 66 can be configured to send drive signals that cause the transducers 16 to generate ultrasound with therapeutic properties. The driver circuit 66 can include a signal generator that generates a signal and a transducer driver that drives the transducers 16 according to the generated signal. In an implementation, the ultrasound generated by the transducers 16 can include low intensity ultrasound (for example, 100 mW/cm2) having a frequency ranging between about 1 and 2 MHz, more particularly about 1.5 MHz. The ultrasound can be pulsed, with a pulse width ranging from about 10 to 2,000 microseconds, more particularly about 200 microseconds, with a repetition frequency ranging from about 0.1 to about 10 KHz, more particularly about 1 KHz.

The communication module 62 can enable the medical device 10 to communicate with another device or system. The communication module 62 can enable communication with a server system, client system, or other computer system over a wired or wireless connection. The communication module 62 can include, for example, a cellular transceiver 70 that can send and/or receive information over a cellular network. The communication module 62 can enable communication over a variety of links and protocols including, for example, 802.11, Bluetooth, Zigbee, and cellular communications protocols.

The interface module 60 can be configured to display information. The interface module 60 can include input and output devices. For example, the interface module 60 can include a screen 72, for example, a liquid crystal display (LCD) or an organic light-emitting diode (OLED) screen. The interface module 60 can also include light-emitting diodes (LEDs) and other indicators. The interface module 60 may include a speaker or other device that can produce sound (not shown), or other output devices. The interface module 60 may also include input capabilities or input devices (not shown), for example, buttons, one or more keypads, and other controls. The screen 72 may be touch-sensitive to receive input from a user.

As described above for the treatment module 58, the interface module 60, the communication module 62, and any other modules can include a storage device with a module identifier that identifies a module. For example, a module identifier may indicate that a particular module is a communication module 62, or more specifically, that the particular module is a communication module 62 of a particular type, and that the module includes a cellular transceiver 70.

Using the module identifier, the processing device 50 can identify a particular module, which can enable the processing device 50 to select control signals, select information to send to the module, and determine what type of information is received from the module. Module profiles for various modules can be stored in the storage device 52. The module profiles can include module identifiers associated with information that enables the processing device 50 to control the various modules. To identify a module connected to the control unit 12, the processing device 50 can (i) receive a module identifier from the module, (ii) access one or more stored module identifiers on the storage device 52, and (iii) match the received module identifier with one of the stored module identifiers. Having identified the module, the processing device 50 can use information in the module profile associated with the module to select information and control signals appropriate for the module. In addition, or alternatively, a module may send additional information to the processing device 50 to indicate parameters of operation and capabilities of the module.

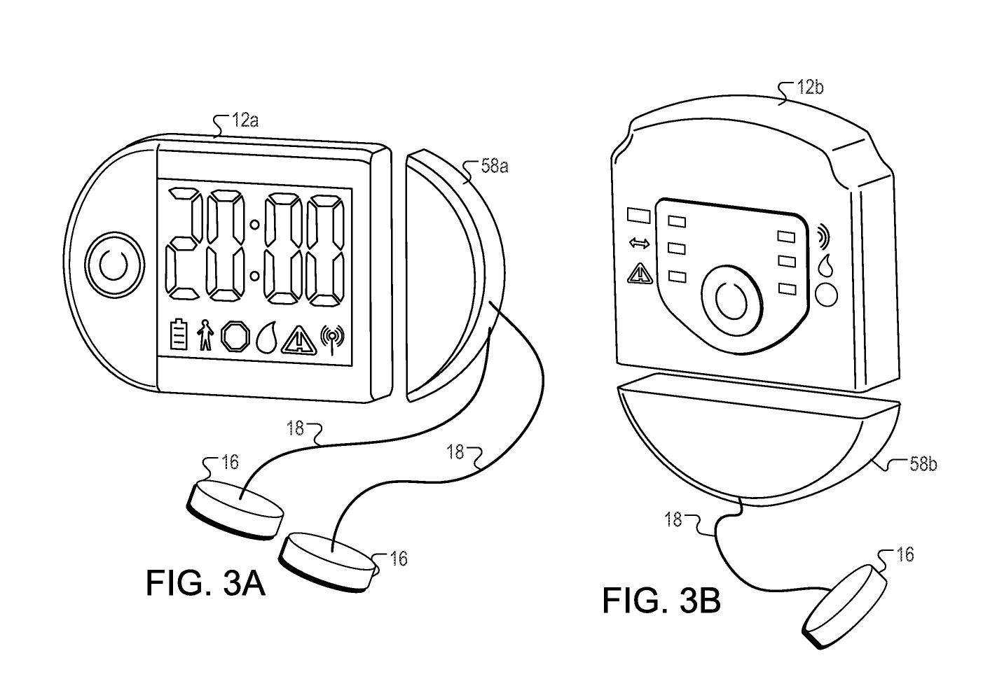

Referring to FIGS. 3A and 3B, control units 12 can vary in appearance and functionality, as illustrated by the differences between the control unit 12a of FIG. 3A and the control unit 12b of FIG. 3B. Control units 12 can differ in size, power output, input and output capabilities, features, and other aspects. For example, control unit 12a may include a screen integrated into the control unit, but the control unit 12b may not. Control unit 12a may also include a higher output power supply 54 than the power supply of control unit 12b. Even though the control units 12a, 12b differ in some respects, the control units 12a, 12b can include identical interfaces to receive and control the treatment modules. The treatment modules may be interchangeable between a fully featured control unit 12a and a basic control unit 12b.

A treatment module 58a-58b can include a housing that extends or completes the exterior surface or shape of the core module housing of the control unit 12. The control unit 12 and the treatment module 58a-58b can be shaped so that the combination of the control unit 12 and the treatment module 58a-58b provides a consistent appearance and feel. For example, the addition of a treatment module 58a-58b to the control unit 12 can complete a surface, line, symmetrical aspect that is present in the assembled combination.

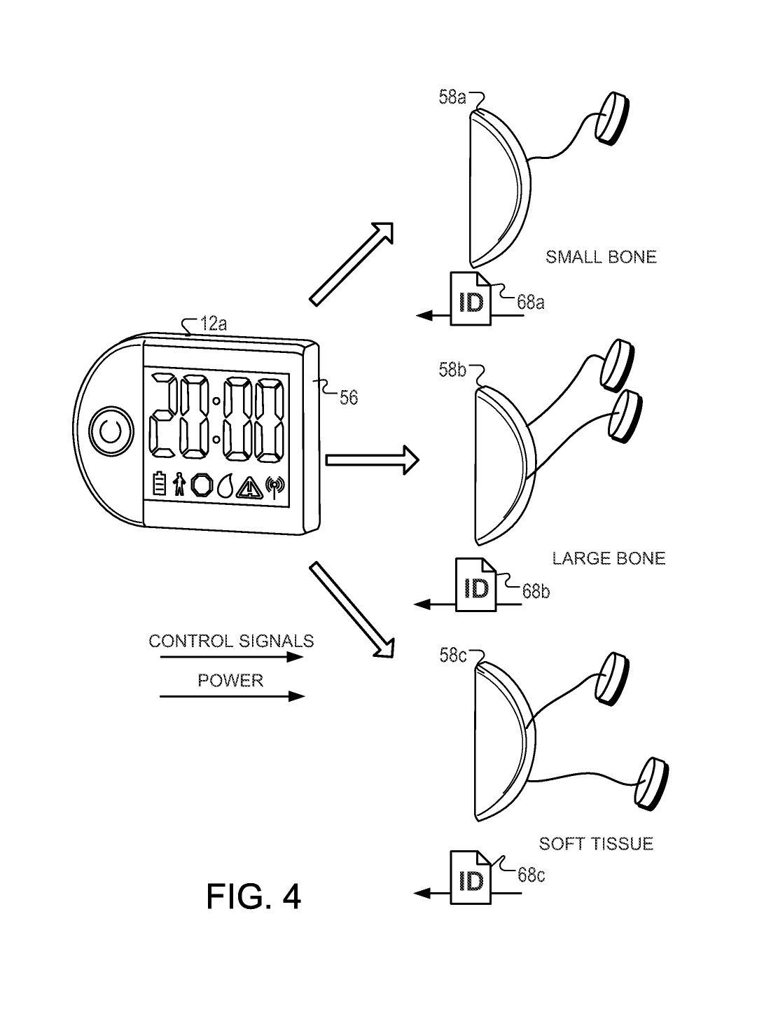

Referring to FIG. 4, as described above, a single control unit 12 can be configured to receive each of several different treatment modules 58a-58c. Each treatment module 58a-58c can be configured to treat a particular health condition or a particular set of health conditions. For example, treatment module 58a may be configured to treat a small broken bone, treatment module 58b may be configured to treat a large broken bone, and treatment module 58c may be configured to treat injured soft tissue. Because the control unit 12 can operate with any of the treatment modules 58a-58c, the control unit 12 can be used in the treatment of a variety of health conditions.

To distinguish the treatment modules 58a-58c, each treatment module 58a-58c can include a different treatment module identifier 68a-68c. Each treatment module 58a-58c can include at least one ultrasound transducer driver circuit 66 and at least one ultrasound transducer 16.

Because treatment modules 58a-58c are configured to treat different health conditions, treatment modules 58a-58c may differ in hardware, functionality, types of input needed (for example control signals from the processing device), types of output produced, and other aspects. For example, treatment modules 58a-58c may differ in the number, size, angle, shape, configuration, application method, or power output of ultrasound transducers 16, among other differences. As another example, the frequency, duration, intensity, pulse characteristics, and pattern of ultrasound produced may vary from one treatment module 58a-58c to another depending on the health conditions the treatment modules 58a-58c are configured to treat.

The processing device 50 of the control unit 12 may identify the particular treatment module 58 that is coupled to the control unit 12. In one implementation, identifying a treatment module can enable the processing unit 50 to send control signals that (i) are compatible with the treatment module 58 and (ii) result in the correct treatment for the health condition that the treatment module 58 is configured to treat.

As an example, the treatment module 58a may be operatively connected to the control unit 12 at an interface 56 of the control unit 12. The processing device 50 of the control unit 12 may detect that the treatment module 58a is connected.

The processing device 50 can determine which particular treatment module, from a plurality of treatment modules (for example, treatment modules 58a-58c), is coupled to the module interface 56. The treatment module 58a can send the treatment module identifier 68a automatically, in response to a request from the processing device 50, periodically, or as part of an initialization process when the treatment module 58a is connected to the control unit 12. The processing device 50 can receive the module identifier 68a, and may store the module identifier 68 in the storage device 52. The processing device 50 can compare the received module identifier 68 with one or more stored module identifiers. The processing device 50 can match the received module identifier 68a with one of the stored module identifiers to identify the treatment module 58a.

Once the treatment module 58a is identified, the processing device 50 can select control instructions for the particular treatment module 58a from a plurality of control instructions. In some implementations, because different treatment modules 58a-58c can be configured to treat different health conditions, control instructions should correspond to the particular treatment module 58a that is coupled to the control unit 12 for treatment to be applied correctly. For example, the processing device 50 may access information about the treatment module 58a that is stored in the storage device 52. The stored information may indicate the types of health conditions the treatment module 58a is configured to treat, the types of control instructions accepted by the treatment module 58a, and/or treatment parameters, for example, the frequency, intensity, duration of ultrasound to be produced or the amount of power required by the treatment module 58a. Different control instructions can be selected to control different treatment modules 58a-58c, thus permitting each of the modules 58a-58c to be controlled to appropriately treat the corresponding health condition(s).

The processing device 50 can control the particular treatment module 58a using the selected control instructions such that an ultrasound assembly of the particular treatment module applies the ultrasound treatment for which the ultrasound assembly is configured. The ultrasound assembly can include at least one ultrasound transducer driver circuit 66 and at least one ultrasound transducer 16.

To treat a health condition that cannot be treated by the treatment module 58a, the treatment module 58a can be disconnected from the control unit 12. Another module, for example the treatment module 58b or the treatment module 58c, can be connected to the control unit 12 to treat the different health condition.

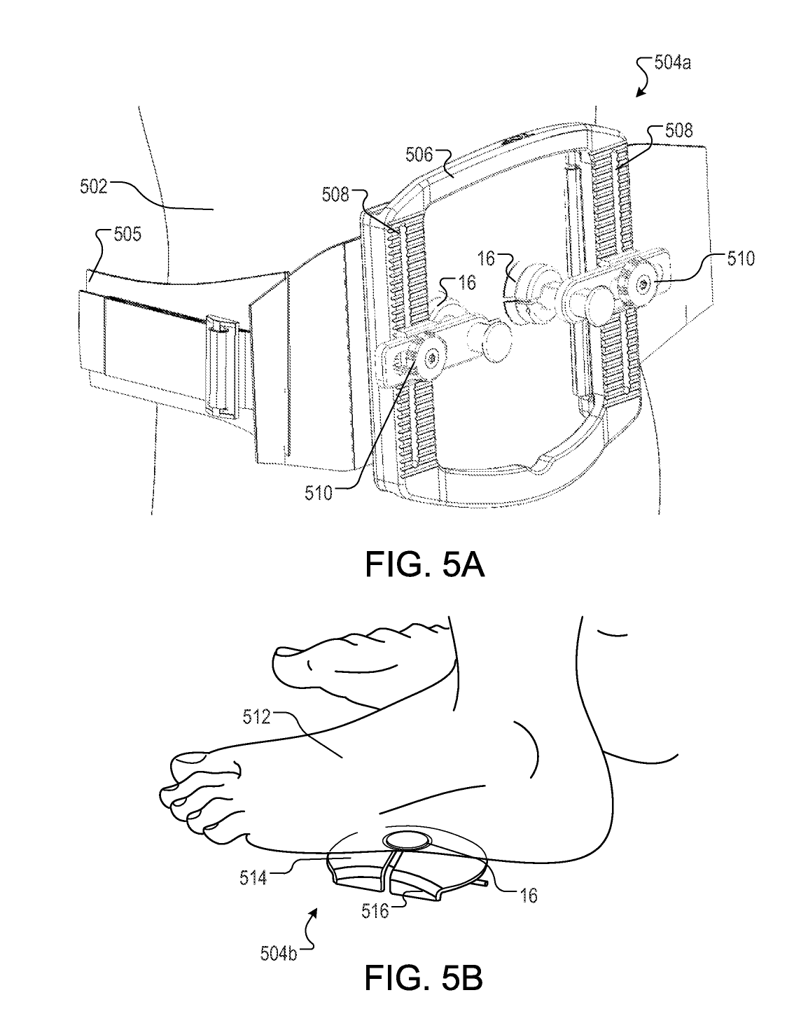

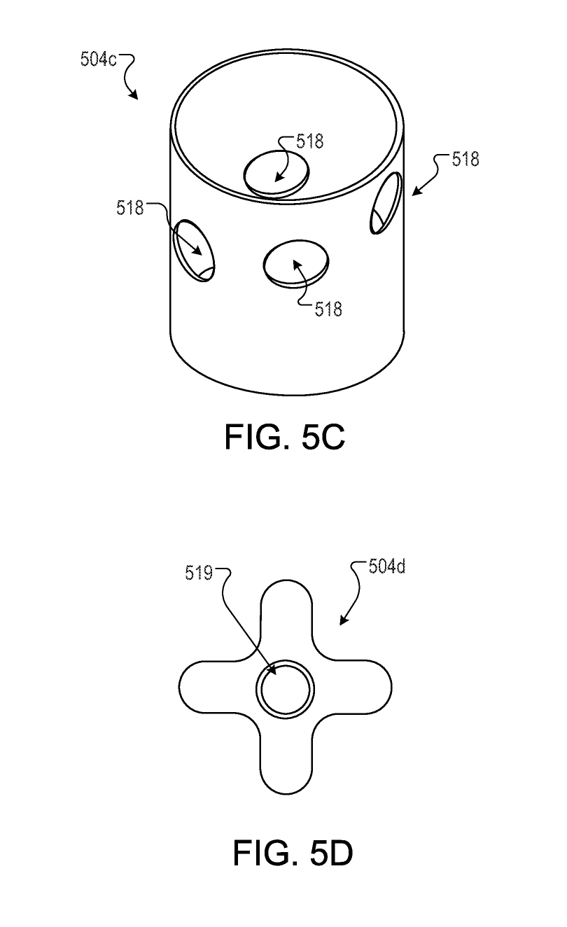

Referring to FIGS. 5A to 5D, treatment modules 58 can include various applicators 504a-504d for treatment of different health conditions. Each of the configurations illustrated may be included in different treatment modules 58, or may be separate from the treatment modules 58. In some cases, an applicator 504a-504d may be compatible with multiple different treatment modules 58.

FIG. 5A illustrates a back applicator 504a that is configured to position two ultrasound transducers 16 at a patient's back 502 or spine. The back applicator 504a can include a strap 505 that attaches the applicator 504a to the patient. The applicator 504a can also include a frame 506 that allows the position of the transducers 16 to be adjusted. For example, the frame 506 may include racks 508 that enable one or more transducers to be positioned on the patient's back 502. In one implementation, the applicator 504a includes controls 510 (for example, a dial or knob) that enable a patient or physician to adjust the position of the transducers 16. The controls 510 may also enable the connection between the transducers 16 and the frame 506 to be loosened and then enable the transducers 16 to be secured in another position.

FIG. 5B illustrates a foot applicator 504b configured to treat a broken bone of a patient's foot 512. The foot applicator 504b can house one or more transducers 16 and can be configured to apply treatment to the underside of the foot 512 while the foot 512 is placed on the applicator 504b. The applicator 504b can maintain the proper orientation of the transducer 16 relative to the foot 512 during treatment. The exterior of the applicator 504b can be shaped to provide a generally smooth contact surface 514, for example, even when a transducer 16 is attached, to enhance the comfort of the patient. The applicator 504b can include one or more alignment features 516 to indicate proper placement of the applicator.

FIG. 5C illustrates a spiral fracture applicator 504c that may be used to treat spiral fractures, for example, spiral fractures of the arm, wrist, or leg. As illustrated, the applicator 504c can define one or more openings 518 that receive one or more transducers 16. The transducers 16 can be placed in the openings 518 that correspond to the health condition of the patient. For example, a physician may select three openings 518 in which transducers 16 should be inserted. The transducers 16 may be secured to the applicator 504c at the openings 518 so the transducers are not accidentally dislodged during treatment.

FIG. 5D illustrates an applicator 504d that may be used to treat a site that is hard to reach, for example, the site of a clavicle fracture. The applicator 504d includes at least one opening 519 that receives a transducer 16. To secure the applicator 504d to the treatment site, the applicator 504d may be weighted or may include an adhesive surface.

The techniques described above are not limited to any particular hardware or software configuration. Rather, they may be implemented using hardware, software, or a combination of both. The methods and processes described may be implemented as computer programs that are executed on programmable computers comprising at least one processor and at least one data storage system. The programs may be implemented in a high-level programming language and may also be implemented in assembly or other lower level languages, if desired.

Any such program will typically be stored on a computer-usable storage medium or device (e.g., EPROM, EEPROM, CD-ROM, ROM, RAM, magnetic disk, flash memory, or embedded memory). When read into the processor of the computer and executed, the instructions of the program cause the programmable computer to carry out the various operations described above. For example, a programmable computer can be a microcontroller and associated data storage contained in a core module housing of a medical device.

A number of implementations have been described. Nevertheless, it will be understood that various modifications may be made. Each of the features described may be implemented individually or in combination or any appropriate sub-combination. In addition, the techniques described above can be used for medical devices other than ultrasound treatment devices. For example, the control unit described or a similar control unit can be used with treatment modules that perform non-ultrasound medical treatments. Accordingly, other implementations are within the scope of the following claims.

1. A modular medical device, comprising:
a core module housing, the core module housing containing:
a treatment module interface configured to couple to any one of a plurality of treatment modules, wherein each of the plurality of treatment modules includes a treatment assembly configured to apply a medical treatment; and
one or more processing devices coupled to the treatment module interface and configured to:
when one of the treatment modules is coupled to the treatment module interface, determine which particular treatment module, from the plurality of treatment modules, is coupled to the treatment module interface;
select, based on the determination, a set of control instructions for the particular treatment module from a plurality of control instructions; and
control the particular treatment module using the selected control instructions such that the treatment assembly of the particular treatment module applies the medical treatment for which the treatment assembly is configured.

2. The modular medical device of claim 1 wherein the treatment assemblies of the plurality of treatment modules are configured to treat different health conditions.

3. The modular medical device of claim 1 wherein:
the treatment assembly of each of the plurality of treatment modules includes an ultrasound assembly configured to apply an ultrasound treatment, and
to control the particular treatment module the one or more processing devices are configured to control the particular treatment module using the selected control instructions such that the ultrasound assembly of the particular treatment module applies the ultrasound treatment for which the ultrasound assembly is configured.

4. The modular medical device of claim 3 wherein each of the ultrasound assemblies includes one or more ultrasound transducers and one or more ultrasound transducer driver circuits.

5. The modular medical device of claim 4 wherein the one or more ultrasound transducers of each of the plurality of treatment modules vary in number, size, angle, or application method to treat a health condition.

6. The modular medical device of claim 3 wherein:
the core module contains one or more data storage devices in communication with the one or more processing devices; and
to select the set of control instructions for the particular treatment module from the plurality of control instructions, the one or more processing devices are configured to access information stored on the data storage device that indicates a frequency, an intensity, or a duration of ultrasound to be produced by the particular treatment module.

7. The modular medical device of claim 1 wherein the core module contains one or more data storage devices in communication with the one or more processing devices, the data storage device storing module profiles for each of the plurality of treatment modules.

8. The modular medical device of claim 1 wherein the core module housing further contains a power supply module configured to supply power over the treatment module interface.

9. The modular medical device of claim 1 wherein:
each of the plurality of treatment modules includes a treatment module identifier; and
the one or more processing devices are further configured to:
receive the treatment module identifier from the particular treatment module.

10. The modular medical device of claim 9 wherein, to determine which treatment module is coupled to the treatment module interface, the one or more processing devices are configured to match the received treatment module identifier with a stored treatment module identifier.

11. The modular medical device of claim 1 wherein each of the plurality of treatment modules includes an applicator configured for treatment of a different health condition.

12. The modular medical device of claim 1 wherein the core module housing further contains a LCD screen and a wireless transceiver.

13. A method performed by one or more processing devices comprising:
determining, by a processing device in a core module housing, which treatment module of a plurality of treatment modules is coupled to a treatment module interface of the core module housing, each of the plurality of treatment modules including a treatment assembly configured to apply a medical treatment;
selecting, based on the determination, a set of control instructions for the particular treatment module from a plurality of control instructions; and
controlling the particular treatment module using the selected control instructions such that the treatment assembly of the particular treatment module applies the medical treatment for which the ultrasound assembly is configured.

14. The method of claim 13 wherein the treatment assemblies of the plurality of treatment modules are configured to treat different health conditions.

15. The method of claim 13 wherein:
each of the plurality of treatment modules includes an ultrasound assembly configured to apply an ultrasound treatment for a particular health condition; and
controlling the particular treatment module using the selected control instructions comprises controlling the particular treatment module using the selected control instructions such that the ultrasound assembly of the particular treatment module applies the ultrasound treatment for which the ultrasound assembly is configured.

16. The method claim 15 wherein each of the ultrasound assemblies includes one or more ultrasound transducers and one or more ultrasound transducer driver circuits.

17. The method claim 16 wherein the one or more ultrasound transducers of each of the plurality of treatment modules vary in number, size, angle, or application method to treat a particular health condition.

18. The method of claim 15 wherein selecting a set of control instructions for the particular treatment module from the plurality of control instructions comprises accessing information stored on a data storage device of the core module housing that indicates a frequency, an intensity, or a duration of ultrasound to be produced by the particular treatment module.

19. The method of claim 13 further comprising supplying power to the particular treatment module over the treatment module interface.

20. The method of claim 13 wherein determining which treatment module of the plurality of treatment modules is coupled to the treatment module interface comprises receiving a treatment module identifier from the particular treatment module.

21. The method of claim 20 wherein determining which treatment module of the plurality of treatment modules is coupled to the treatment module interface comprises matching the received treatment module identifier with a stored treatment module identifier.

22. The method of claim 13 wherein each of the plurality of treatment modules includes an applicator configured for treatment of a different health condition.

23. A modular medical device for ultrasound therapy, comprising:
a core module housing, the core module housing containing:
a treatment module interface configured to couple to any one of a plurality of treatment modules, wherein each of the plurality of treatment modules includes an ultrasound assembly configured to apply an ultrasound treatment for a particular health condition; and
one or more processing devices coupled to the treatment module interface and configured to:
when one of the treatment modules is coupled to the treatment module interface, determine which particular treatment module, from the plurality of treatment modules, is coupled to the treatment module interface;
select, based on the determination, a set of control instructions for the particular treatment module from the plurality of control instructions; and
control the particular treatment module using the selected control instructions such that the ultrasound assembly of the particular treatment module applies the ultrasound treatment for which the ultrasound assembly is configured.

24. A method performed by one or more processing devices comprising:
determining, by a processing device in a core module housing, which treatment module of a plurality of treatment modules is coupled to a treatment module interface of the core module housing, each of the plurality of treatment modules including an ultrasound assembly configured to apply an ultrasound treatment for a particular health condition;
selecting, based on the determination, a set of control instructions for the particular treatment module from a plurality of control instructions; and
controlling the particular treatment module using the selected control instructions such that an ultrasound assembly of the particular treatment module applies the ultrasound treatment for which the ultrasound assembly is configured.
